(19) United States
(12) Patent Application Publication
(10) Pub. No.: US 2025/0066833 A1
(43) Pub. Date: Feb. 27, 2025

(54) REAL-TIME MONITORING OF MICROBIAL GROWTH IN WATER FLUID WELLS

(71) Applicant: SAUDI ARABIAN OIL COMPANY, Dhahran (SA)

(72) Inventors: Abdulmohsen A. AL-HUMAM, Dhahran (SA); Manar ALAHMARI, Dhahran (SA)

(73) Assignee: SAUDI ARABIAN OIL COMPANY, Dhahran (SA)

(21) Appl. No.: 18/455,222

(22) Filed: Aug. 24, 2023

(51) Int. Cl. C12Q 1/04; G01N 21/76

(52) U.S. Cl. CPC C12Q 1/04; G01N 21/763

(57) ABSTRACT

Methods for monitoring total microbial concentration in a water fluid well comprising water-based fluid. The methods include collecting a water-based fluid sample from the water fluid well; mixing the sample with a liquid reagent comprising luciferase enzyme; measuring photon release of the sample-liquid reagent composition mixture in a luminometer; determining the total microbial concentration of the water-based fluid in the water fluid well; assigning an ATP-based action level based on the total microbial concentration of the water-based fluid in the water fluid well; and performing an action based on the assigned ATP-based action level.

FIELD OF THE DISCLOSURE

Embodiments in accordance with the present disclosure generally relate to methods for real-time, onsite monitoring of total microbial presence in water fluid wells; in particular, the present disclosure relates to methods for real-time, onsite monitoring of total microbial presence in water disposal wells, water injection wells, and water supply wells.

### BACKGROUND

Controlling microbial growth in water fluid wells, such as water disposal wells, water injection wells, and water supply wells, is essential for effective functioning of such wells.

Microbial growth refers to contamination with bacteria. In water, bacteria can be single-celled or multi-celled and are generally ubiquitous. Microbial growth can be sustained with a wide range of nutrients, both organic and inorganic.

Various water fluid wells (or simply “water wells”) are used in the oil and gas industry for various purposes including, for example, disposal of water-based (e.g., fresh water, brine) used for various operations, such as drilling, fracturing, and other well treatment fluids; injection of water fluids into hydrocarbon wells as part of enhanced oil recovery operations; and supply water for various operations, such as drilling and fracturing. Microbial growth in of these wells can cause a number of issues including, but not limited to, bacterial-induced corrosion of metal-based engineering materials (e.g., well piping, well casing, and the like), near-well formation pugging or damage, groundwater or surface contamination, environmental hazards (e.g., increased H₂S), and harm to flora and fauna, including humans.

Microbial growth in water fluid wells often requires costly remedial measures, such as the use of various chemicals that are designed to kill bacteria. Moreover, if microbial growth goes unchecked for an extended period of time, these remedial costs amplify. Further, in some instances, a water well must be abandoned if the microbial growth is substantial and remedial efforts prove ineffective.

In view of the foregoing, the present disclosure provides real-time methods of monitoring microbial activity to permit timely remedial treatments (e.g., biocides) to control microbial growth levels.

### SUMMARY OF THE DISCLOSURE

Embodiments in accordance with the present disclosure generally relate to methods for real-time, onsite monitoring of total microbial presence in water fluid wells; in particular, the present disclosure relates to methods for real-time, onsite monitoring of total microbial presence in water disposal wells, water injection wells, and water supply wells.

According to an embodiment consistent with the present disclosure, a method for monitoring total microbial concentration in a water fluid well comprising water-based fluid is provided, the method including collecting a water-based fluid sample from the water fluid well; mixing the sample with a liquid reagent comprising luciferase enzyme, thereby forming a sample-liquid reagent composition; measuring photon release of the sample-liquid reagent composition mixture in a luminometer, wherein the photon release is proportional to an amount of adenosine-5′-triphosphate (ATP) in the sample, and the amount of ATP in the sample is proportional to the total microbial concentration of the water-based fluid in the water fluid well; determining the total microbial concentration of the water-based fluid in the water fluid well; assigning an ATP-based action level based on the total microbial concentration of the water-based fluid in the water fluid well; and performing an action based on the assigned ATP-based action level.

Any combinations of the various embodiments and implementations disclosed herein can be used in a further embodiment, consistent with the disclosure. These and other aspects and features can be appreciated from the following description of certain embodiments presented herein in accordance with the disclosure and the accompanying drawings and claims.

### BRIEF DESCRIPTION OF THE DRAWINGS

The following figures are included to illustrate certain aspects of the embodiments, and should not be viewed as exclusive embodiments. The subject matter disclosed is capable of considerable modifications, alterations, combinations, and equivalents in form and function, as will occur to those skilled in the art and having the benefit of this disclosure.

FIG. 1 provides is a flowchart illustrating a method for ATP-bioluminescence testing of a water-based fluid from a water fluid well, according to one or more aspects of the present disclosure.

FIG. 2 shows the linear correlation of ATP concentration versus colony forming units (CFU) in a representative water-based fluid from a water fluid well, according to one or more aspects of the present disclosure.

### DETAILED DESCRIPTION

Embodiments in accordance with the present disclosure generally relate to methods for real-time, onsite monitoring of total microbial presence in water fluid wells; in particular, the present disclosure relates to methods for real-time, onsite monitoring of total microbial presence in water disposal wells, water injection wells, and water supply wells.

Reference is made below to various embodiments of the disclosed subject matter, examples of which are illustrated in part in accompanying drawings. While the disclosed subject matter will be described in conjunction with the enumerated claims, it will be understood that the exemplified subject matter is not intended to limit the claims to the disclosed subject matter.

Water plays a large role in various oil and gas operations. For example, wells are drilled into subterranean formations for oil and gas recovery, requiring the use of circulating fluids to enable the drilling process and carry cuttings and rock fragments to the surface. These wells may be stimulated, such as by hydraulic fracturing, in which a treatment fluid is injected into the subterranean formation from a well at a rate and pressure to induce fractures through which oil and gas can flow. Enhanced oil recovery may be implemented to increase production of a well by injecting fluids to increase or maintain pressure therein and drive oil and gas to the surface for recovery. Finally, any or all of these fluids may be composed of water-based fluids, which may or may not comprise various additional elements, such as salts and other chemicals associated with oil and gas operations.

A variety of microorganisms may be present in disposal, injection, or supply water-based fluids (collectively “oil and gas water-based fluid” or “OG water-based fluids”) including, but not limited to, sulfate-reducing prokaryotes (SRP) (e.g., sulfate-reducing bacteria (SRB) and sulfate-reducing archaea (SRA)), as well as other subterranean formation and surface microorganisms (e.g., slime-forming bacteria, iron-oxidizing bacteria, and the like), and any combination thereof. The effect of excessive microbial growth may result in microbiologically influenced corrosion (MIC) of oil and gas metal-based engineering equipment (e.g., pipeline, production or processing tubulars, and the like), biofouling of the well, biomass plugging in the formation, and the metabolism of SRP producing hydrogen sulfide (H₂S) that can sour the well and produce harm to the environment, among the other effects discussed herein. These effects ultimately result in shorter water fluid well lifespans and equipment associated therewith, and potentially total abandonment of a water fluid well.

Standard oil and gas practices to monitor microbial growth rely on culture-dependent techniques, such as use the Most Probable Number (“MPN”) test to monitor microbial growth. The MPN test provides quantitative estimation of microbial growth based on triplicate serial dilution of a microbial culture. Although NACE International recognizes this and other culture-dependent techniques as the standard in oilfield systems monitoring (NACE TM0194 2014), culture-dependent techniques often underestimate the total microbial population and further require substantial time dedication, where analysis ranges from 24 hours to 28 days, for example. Common contaminate SRB takes at least 10 to 14 days of incubation before accurate microbial quantification (count) can be made. This delay in results turnover adversely affects timely remedial response actions, and allows the bacterial growth and resilience to increase.

Differently, the various embodiments of the present disclosure, microbial growth in water fluid wells is monitored to quantify and assess necessary remedial actions using a real-time, portable adenosine-5′-triphosphate (ATP)-bioluminescence assay that can be used onsite for sampling over a predetermine time period. Such water fluid wells include water disposal wells, water injection wells, and water supply wells, each for use in or related to oil and gas operations. Monitoring microbial growth in these wells advantageously allows avoidance of costly remedial measures associated with substantial microbial growth as described above. Indeed, each of these water fluid wells require tight control of bacterial activity before disposal, injection, or supply of the water-based fluids as part of a particular operation.

Advantageously, the various methods of the present disclosure provide early warning insight of microbial grown in water fluid wells using ATP-bioluminescence including, but not limited to, measuring free-living bacteria and archaea, particle-associated bacteria and archaea, bacterial and archaeal biofilm, any combination thereof, indicative of the general hygienic nature of a water fluid well; MIC activity, where high ATP values indicate the presence of an environment that is conducive to corrosion via microbial growth; and water fluid well souring due to the production of H₂S from microbial growth.

### Definitions

As used herein, the terms “microbial,” “microorganism,” and “microbe,” and grammatical variants thereof, each refer to bacterial microorganisms, such as those described herein, including sulfate-reducing prokaryotic bacteria.

As used herein, the terms “microbial growth,” and grammatical variants thereof, refers to presence and proliferation (increase in number) of microbes in a water-based fluid, as described herein, and encompasses the term “microbial contamination,” and grammatical variants thereof.

As used herein, the term “total microbial concentration,” and grammatical variants thereof, refers to the total concentration (amount) of microbes within a particular water-based fluid in a water fluid well.

As used herein, the term “water-based fluid,” and grammatical variants thereof, refers to fresh water, saltwater (e.g., water containing one or more salts dissolved therein), brine (e.g., saturated salt water), seawater, produced water, wastewater (e.g., from hydraulic fracturing activities), treated wastewater, and any combination thereof. Chemicals or other components may be mixed with or otherwise associated with the water-based fluid, without departing from the scope of the present disclosure. As provided above, the term “oil and gas water-based fluid,” or “OG fluid,” and grammatical variants thereof, is used to refer to water-based fluids used with reference to water disposal wells, water injection wells, and water supply wells.

As used herein, the term “water disposal well,” and grammatical variants thereof, refers to a well in which waste water-based fluids from oil and gas operations are injected for safe disposal (e.g., to avoid contamination of freshwater aquifers). Often, a water disposal well is a depleted oil or gas well.

As used herein, the term “water injection well,” and grammatical variants thereof, refers to a well in which water-based fluids are injected (rather than produced), with the primary objective typically being to maintain reservoir pressure. Water injection wells are generally used to inject water-based fluids (e.g., filtered and treated seawater) into a hydrocarbon well to pressurize and displace hydrocarbons during enhanced oil recovery operations.

As used herein, the term “water supply well,” and grammatical variants thereof, refers to a well in which water-based fluids are stored for use in oil and gas operations, such as drilling, fracturing, acidizing, other stimulation operations, enhanced oil recovery, and the like, and any combination thereof. The source of the water-based fluid within a water supply well may include, for example, either shallow fresh water or deep formation water.

### ATP-Bioluminescence Methods of Monitoring Microbial Growth in Water Fluid Wells

The present disclosure provides methods for real-time, rapid, portable testing of active microbes using ATP-bioluminescence. ATP-bioluminescence accurately identifies metabolically active cells in a given sample, measuring available ATP (i.e., cellular ATP, or cATP).

The ATP-bioluminescence tests described herein are based on the detection of light generated by a chemical reaction within a water-based fluid sample from a water fluid well. The enzyme luciferase catalyzes ATP-mediated oxidation of luciferin to form oxyluciferin. In doing so, the ATP is dephosphorylated to adenosine monophosphate (AMP) and pyrophosphate, in turn releasing a photon of light. This light (bioluminescence) is proportional to the amount of ATP in the sample, which equates to the quantity of microbes within the tested sample.

ATP-bioluminescence testing benefits from the portability of test kits and rapid, real-time (onsite) monitoring of microbial contamination at water fluid wells. These water fluid wells include water disposal wells, water injection wells, and water supply wells. Recent 2^{nd} generation ATP-bioluminescence test kits have overcome specific interference substances related to the oil and gas industry to further boost accuracy. Due to the expediency and real-time capabilities of the method described herein, microbial growth mitigation in water fluid wells can be acted on in a timely manner.

In one or more embodiments, the ATP-bioluminescence test methods of the present disclosure for monitoring microbial growth water fluid wells include collecting a water-based fluid sample from a water fluid well. The water fluid well may include a water disposal well, a water injection well, or a water supply well. The sample of water-based fluid is mixed with a liquid reagent comprising luciferase enzyme to form a sample-liquid reagent composition. The liquid reagent may comprise other components, such as a buffer. Using a luminometer, photon release (light) of the sample-liquid reagent is measured, wherein the photon release is proportional to an amount of ATP in the sample and the amount of ATP in the sample is proportional to the total microbial concentration of the water-based fluid in the water fluid well.

An ATP-based action level may be assigned to the water-based fluid and, thus, the particular water fluid well, based on the total microbial concentration. Because the ATP-bioluminescence methods of the present disclosure are performed in real-time, on-site, a particular ATP-based action level can be assigned in real-time. The ATP-based action levels generally include assessment of the total microbial concentration and a determination of whether the water-based fluid in the water fluid well is in good control (thus, requiring action by continued monitoring) or whether a timely intervention-type action should be taken. Thus, the methods of the present disclosure include taking a good control action by continuing monitoring, or preventative action or corrective action, wherein the preventative action is lesser in degree (e.g., a comparatively reduced amount of biocide applied) compared to the corrective action. Moreover, as with the assignment of the ATP-based action level, preventative or corrective actions may be performed in real-time, on-site immediately upon determining the ATP-based action level and further tailored thereon.

The ATP-based action levels are based on a strong correlation between microbial numbers and ATP concentration. Microbial control in these water fluid wells is critical to protect equipment, environment, human health, and industry costs, as described above. For water-based fluid in these water fluid wells, the ATP-based action levels may be based on pg of ATP/mL (see the Example below), as provided in Table 1.

TABLE 1

| | Good Control | Preventative Action | Corrective Action |
|---|---|---|---|
| Water-Based | <25 | 25-200 | >200 |
| Fluid | pg of ATP/mL | pg of ATP/mL | pg of ATP/mL |

Accordingly, a water-based fluid sample from a water fluid well is in good control if it is within the specified good control limit (25 pg of ATP/mL); a water-based fluid sample from a water fluid well requires preventative action if it is higher than the specified good control limit and lower than the specified corrective action limit (25-200 pg of ATP/mL); and a water-based fluid sample from a water fluid well requires corrective action if it is higher than the preventative action limit and, thus, significantly higher than the good control limit (>200 pg of ATP/mL).

Various actions may be taken depending upon the assignment of a particular ATP-based action level to a sample of water-based fluid collected from a water fluid well and, additionally as assessed by field engineers on-site at the water fluid well. Examples of suitable actions include ATP-bioluminescence monitoring, ATP-bioluminescence re-testing, APT-bioluminescence monitoring frequency increase, APT-bioluminescence microbial source identification, and in the event of significantly high microorganism activity, biocide selection and treatment. Actions taken based on an assigned ATP-based action level for use in the embodiments of the present disclosure are provided in Table 2.

TABLE 2

| ATP-Based | |
|---|---|
| Action Level | Action |
| Good Control | Continue ATP-Bioluminescence Monitoring |
| Preventative | Re-Test Sample and Increase Monitoring Frequency |
| Action | and/or Proactively take Corrective Action via Biocide |
| | Section and Treatment |
| Corrective | Re-Test Sample and Identify Source of Microbial |
| Action | Contamination and Take Corrective Action via Biocide |
| | Selection and Treatment |

As provided in Table 2, one or both of the preventative and/or corrective actions may include, but are not limited to, biocide selection and treatment (application). Suitable biocides may include, but are not limited to, oxidizing biocides, non-oxidizing biocides, and any combination thereof. Examples of oxidizing biocide include, but are not limited to, chlorine dioxide, sodium hypochlorite, bromine peroxide, hydrogen peroxide, and the like, and any combination thereof. Examples of non-oxidizing biocides include, but are not limited to, 2,2-dibromo-3-nitrilopropionamide (DBNPA), tetrakis(hydroxymethyl)phosphonium sulfate (THPS). glutaraldehyde, and the like, and any combination thereof. Depending on the particular total microbial concentration and the selected biocide, a biocide may be applied in the range of about 1% by volume (vol %) to about 20 vol %, encompassing any value and subset therebetween, such as about 1 vol % to about 5 vol %, or about 5 vol % to about 10 vol %, or about 10 vol % to about 15 vol %, or about 15 vol % to about 20 vol %.

The methods of the present disclosure advantageously allow on-site, rapid microbial growth (and concentration) monitoring, thereby permitting testing personnel (e.g., field engineers) to monitor microbial activity in real-time at selected intervals (e.g., pre-determined time intervals) or on-the-fly and to react swiftly to perform remedial actions, such as selection of appropriate biocide type(s) and application frequency, thus reducing excessive biocide use. Remedial action regimens or long-term treatment regimens can be adjusting in a timely manner using the methods of the present disclosure. Indeed, microbial monitoring may be performed before and after biocide treatment to determine the effectiveness of a particular biocide(s) and to make any necessary adjustments. Moreover, the methods of the present disclosure can be used to perform root cause investigation to determine one or more sources of microbial growth (e.g., by sampling at different locations and comparing the microbial concentration at each location, where a greater concentration is indicative of a source). Sources of microbial contamination may include, but are not limited to, a filter, a heat exchanger, a length of flow pipe, and the like, and any combination thereof.

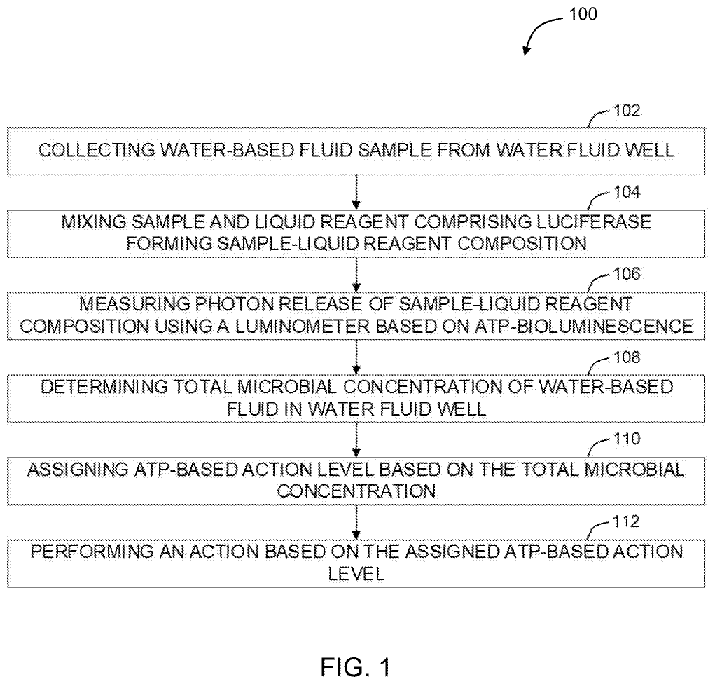

Referring now to FIG. 1, provided is a flowchart illustrating a method 100 for monitoring total microbial concentration of a water-based fluid in a water fluid well using ATP-bioluminescence testing, according to one or more aspects of the present disclosure. The method 100 includes, at 102, collecting a water-based sample from a water fluid well. At 104, the method 100 includes mixing the sample with a liquid reagent comprising luciferase enzyme, thereby forming a sample-liquid reagent composition. The mixing at 104 results in a reaction in which, due to the presence of viable microbes in the sample-liquid reagent composition (i.e., produce ATP), the luciferase enzyme catalyzes ATP-mediated oxidation of luciferin to form oxyluciferin; the ATP is dephosphorylated to AMP and pyrophosphate, in turn releasing a photon of light. As provided herein, the photon release is proportional to an amount of ATP in the sample and the amount of ATP in the sample is proportional to the total microbial concentration of the water-based fluid in the water fluid well. The method 100 further includes, at 106, measuring the photon release of the sample-liquid reagent composition using a luminometer based on ATP-bioluminescence and, at 108, determining the total microbial concentration of the water-based fluid in the water fluid well. At 110, the method 100 includes assigning an ATP-based action level on the total microbial concentration of the water-based fluid in the water fluid well, and at 112, performing an action based on the assigned ATP-based action level.

With continued reference to FIG. 1, when a preventative or corrective action requires re-testing of the water-based sample, it requires repeating at least steps 104, 106, 108. Steps 110 and 112 may additionally be repeated if a substantially different result is obtained upon re-testing, which may be indicative of an anomalous error in the test. That is, the re-testing of the sample can verify the validity of the original results or necessitate further re-testing to ensure correct (reproducible) total microbial concentration measurements are obtained. Similarly, if the ATP-based action level is a good control ATP-based action level, continued monitoring of the total microbial contamination of the water-based fluid in the water fluid well requires repeating steps 104, 106, 108, 110, and 112.

### Nonlimiting Example Embodiment

Embodiments disclosed herein include:

Embodiment A: A method for monitoring total microbial concentration in a water fluid well comprising water-based fluid, the method comprising: collecting a water-based fluid sample from the water fluid well; mixing the sample with a liquid reagent comprising luciferase enzyme, thereby forming a sample-liquid reagent composition; measuring photon release of the sample-liquid reagent composition mixture in a luminometer, wherein the photon release is proportional to an amount of adenosine-5′-triphosphate (ATP) in the sample, and the amount of ATP in the sample is proportional to the total microbial concentration of the water-based fluid in the water fluid well; determining the total microbial concentration of the water-based fluid in the water fluid well; assigning an ATP-based action level based on the total microbial concentration of the water-based fluid in the water fluid well; and performing an action based on the assigned ATP-based action level.

Embodiment A may have one or more of the following additional elements:

Element 1: wherein the water fluid well is a water disposal well.

Element 2: wherein the water fluid well is a water injection well.

Element 3: wherein the water fluid well is a water supply well.

Element 4: wherein the water-based fluid is selected from the group consisting of fresh water, saltwater, brine, seawater, produced water, wastewater, treated wastewater, and any combination thereof.

Element 5: wherein the luminometer is portable and collecting the sample, mixing the sample, measuring the photon release, assigning the ATP-based action level, and performing the action is performed in real-time, on-site at the water fluid well.

Element 6: wherein the total microbial concentration comprises free-living bacteria, free-living archaea, particle-associated bacteria, particle-associated archaea, bacterial biofilm, archaeal biofilm, and any combination thereof.

Element 7: wherein the total microbial concentration comprises sulfate-reducing bacteria.

Element 8: wherein the total microbial concentration comprises sulfate-reducing archaea.

Element 9: wherein the photon release measurement has a repeatable relative standard deviation in the range of 7.4% to 12.4%.

Element 10: further comprising filtering the sample prior to the mixing.

Element 11: wherein the assigned ATP-based action level is a corrective action ATP-based action level, or a corrective action ATP-based action level, and the action comprises at least repeating at least the steps of collecting, mixing, measuring, and determining based on a subsequent water-based fluid from the water fluid well to determine the total microbial concentration of the water-based fluid in the water fluid well.

Element 12: wherein the total microbial concentration of the water-based fluid in the water fluid well is less than 25 pg/ATP mL, and the assigned ATP-based action level is a good control ATP-based action level.

Element 13: wherein the total microbial concentration of the water-based fluid in the water fluid well is less than 25 pg/ATP mL, the assigned ATP-based action level is a good control ATP-based action level, and the action comprises repeating the steps of collecting, mixing, measuring, determining, assigning, and performing based on a subsequent water-based fluid from the water fluid well to determine the total microbial concentration of the water-based fluid in the water fluid well.

Element 14: wherein the total microbial concentration of the water-based fluid in the water fluid well is in the range of 25 pg/ATP mL to 200 pg/ATP mL, and the assigned ATP-based action level is a preventative action ATP-based action level.

Element 15: wherein the total microbial concentration of the water-based fluid in the water fluid well is in the range of 25 pg/ATP mL to 200 pg/ATP mL, the assigned ATP-based action level is a preventative action ATP-based action level, and the action comprises increasing a frequency of monitoring the total microbial concentration of the water-based fluid in the water fluid well.

Element 16: wherein the total microbial concentration of the water-based fluid in the water fluid well is in the range of 25 pg/ATP mL to 200 pg/ATP mL, the assigned ATP-based action level is a preventative action ATP-based action level, and the action comprises introducing a biocide into the water fluid well.

Element 17: wherein the total microbial concentration of the water-based fluid in the water fluid well is greater than 200 pg/ATP mL, and the assigned ATP-based action level is a corrective action ATP-based action level.

Element 18: wherein the total microbial concentration of the water-based fluid in the water fluid well is greater than 200 pg/ATP mL, the assigned ATP-based action level is a corrective action ATP-based action level, and the action comprises introducing a biocide into the water fluid well.

Element 19: wherein the total microbial concentration of the water-based fluid in the water fluid well is greater than 200 pg/ATP mL, the assigned ATP-based action level is a corrective action ATP-based action level, and the action comprises introducing a biocide into the water fluid well, wherein the biocide is selected from the group consisting of an oxidizing biocide, a non-oxidizing biocide, and any combination thereof.

Element 20: wherein the total microbial concentration of the water-based fluid in the water fluid well is greater than 200 pg/ATP mL, the assigned ATP-based action level is a corrective action ATP-based action level, and the action comprises identifying a source of microbial contamination, the source selected from the group consisting of a filter, a heat exchanger, a length of flow pipe, and the like, and any combination thereof.

Embodiment A may have one, more or all of the following Elements in any combination, without limitation: Elements 1, 4-13; Elements 1, 4-11, 14-16; Elements 1, 4-11, 17-20; Elements 2, 4-13; Elements 2, 4-11, 14-16; Elements 2, 4-11, 17-20; Elements 3, 4-13; Elements 3, 4-11, 14-16; Elements 3, 4-11, 17-20.

To facilitate a better understanding of the embodiments of the present invention, the following examples of preferred or representative embodiments are given. In no way should the following examples be read to limit, or to define, the scope of the invention.

### EXAMPLE

In this Example, the use of ATP-bioluminescence test methods were validated for linearity and repeatability for detecting microbial growth in water disposal, injection, and supply wells.

In this Example, a luminometer was used to detect relative light units (RLU) (or photons) released during an ATP-bioluminescence test. A suitable luminometer for use in the methods of the present disclosure includes the PHOTONMASTER™ Luminometer (LuminUltra, New Brunswick, Canada), a portable luminometer that can be used for on-site microbial growth analysis in water fluid wells. The PHOTONMASTER™ is designed to operate with LuminUltra 2^{nd} generation ATP Quench-Gone Organic Modified (QGO-M) test kits that yield results in less than about 10 minutes, including in the range of about 3 minutes to about 10 minutes, or about 5 minutes to about 10 minutes, encompassing any value and subset therebetween.

The ATP QGO-M kits are designed for water-based fluid in water fluid wells including. produced water, injection water, supply water, wastewater, and other water-based treatment fluids (e.g., drilling fluids, fracturing fluids, and the like).

Representative water-based fluid (RWBF) samples were obtained and evaluated as representative of water-based fluid collected from a water supply well. The RWBF samples were spiked with a mixed bacteria population at a concentration of 10³ to 10⁵ cell/mL, encompassing any value and subset therebetween, isolated from the original corresponding sample. Serial dilutions were performed by diluting the spiked RWBF samples in sterile water. The diluted RWBF samples were sterilized by autoclaving at 121° C. for 15 minutes. The diluted RWBF samples were analyzed using both a plate count method and an ATP bioluminescence assay described herein.

Plate Count Method: A plate counter method (PCM) was used to quantify the cultivable (i.e., viable) microbes in the RWBF samples. Results were correlated to ATP-bioluminescence measurements in the same RWBF sample. Tryptic soy agar (TSA) medium was adjusted to a pH of 7-10 and poured into agar plates. After the TSA medium solidified, 0.1 mL samples of the serial diluted RWBF samples were spread on the plates. TSA with a pH of 7 was used for the RWBF samples. Plates were inoculated in triplicate for each RWBF sample dilution and incubated at 37° C. for 18 hours. The average number of colony formation units (CFU) was multiplied by the dilution factor to determine the number of microbes per mL of the RWBF sample (CFU/mL).

ATP-Bioluminescence Assay: RWBF samples were prepared for ATP quantification using the LuminUltra QGO-M test kit. The RWBF samples were analyzed in 20 mL aliquots. After mixing the RWBF samples to ensure homogeneity, the appropriate amount of RWBF sample was taken up with a 20 mL syringe and pushed through a 2 μm filter. After the full volume of the RWBF sample was filtered, the filter was washed by adding 5 mL of a wash solution (LUMICLEAN™, LuminUltra) to the syringe barrel and pushing the wash solution through the filter. Next, the filter was dried by removing the filter from the 20 mL syringe and attaching the filter to a 60 mL syringe. The 60 mL syringe was used to push approximately 60 mL of air through the filter.

The microbes were then extracted from the filter by reattaching the filter to a 20 mL syringe barrel. Next, 1 mL of a lysing agent (ULTRALYSE™ 7, LuminUltra) was added to the barrel of the syringe and pushed through the filter and collected in a 9 mL dilution tube containing a dilution buffer (ULTRALUTE™, LuminUltra).

Thereafter, 100 μL from the dilute tube was added to an assay tube. 100 μL of luciferase enzyme (obtained from LUMINASE™ (LuminUltra)) was added to the RWBF sample, swirled gently five times, and immediately inserted into a luminometer for measurement.

RLU results were converted to picograms (pg) of ATP/mL via the following Equation 1:

$$( \frac{{RLU}_{sample}}{{RLU}_{standard} * {Volume}_{sample}} ) * 10 , 000 = cAPT ( \frac{pg ATP}{mL} ) , \qquad Equation 1$$

where the RLU_{standard} is the RLU generated from reacting the 0.1 mL of reference solution containing 1,000 pg ATP/mL with 0.1 mL of luciferase enzyme reagent. Volumeₛₐₘₚₗₑ is the volume of sample used for the test in mL, and factor 10,000 is derived from the 10-fold dilution of the extracted ATP times the conversion of nanograms (ng) to pg (1,000 pg/ng).

Analysis: The repeatability of the ATP-bioluminescence measurements was tested at various dilutions of the RWBF samples in triplicate. At each dilution, the mean of the ATP concentration was calculated and the relative standard deviation (RSD) was determined by the following Equation 2:

$$RSD = 100 * \frac{S}{\vert \overline{x} \vert} \qquad Equation 2$$

where S is the sample standard deviation (SD) and x is the ATP concentration mean.

Results-Relative Standard Deviations: The repeatability of the ATP-bioluminescence method for quantifying cATP in the RWBF samples was assessed in triplicate by testing seven dilutions thereof. The results are provided in Table 3 below.

TABLE 3

| Dilution of RWBF | Mean cATP | | RSD |
|---|---|---|---|
| Sample | (pg/mL) | SD | (%) |
| 1:1 | 2714 | 45 | 2 |
| 1:2.5 | 996 | 146 | 15 |
| 1:5 | 647 | 81 | 12 |
| 1:25 | 121 | 12 | 10 |
| 1:50 | 53 | 7 | 13 |
| 1:250 | 10 | 1.9 | 18 |
| 1:2500 | 2 | 0.5 | 17 |

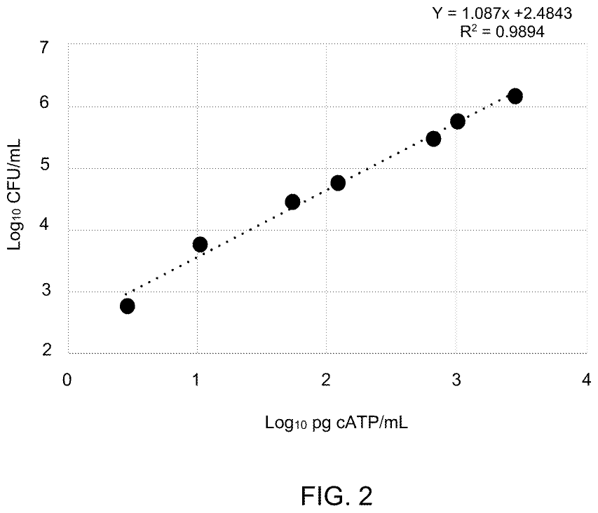

As shown, the ATP-bioluminescence methods of the present disclosure can detect as low as 2 pg/mL of ATP in a water-based fluid sample diluted by 2500 fold. The average RSD of ATP measurements for the seven RWBF sample dilutions having mean cATP between 2 pg/mL and 2714 pg/mL of cATP shown in FIG. 2 was 12.4%. The ATP-bioluminescence methods of the present disclosure are, accordingly, highly repeatable, showing ATP measurements of water-based fluid with average RSD ranging from about 7.4% to about 12.4%, encompassing any value and subset therebetween.

### Results-Correlation Between CFU and ATP-Bioluminescence Testing

The linearity of the ATP-bioluminescence measurements and assessed by plotting the log₁₀ of the ATP concentration of each dilution RWBF sample. A correlation between the measured ATP concentration (log₁₀ pg ATP/mL) and the number of cultivable microbial cells (log₁₀ CFU/mL) was determined. FIG. 2 shows the linear correlation between the ATP-bioluminescence method and the CFU method for determining microbial growth in a water-based fluid within a water fluid well, including a water disposal well, a water injection well, or a water supply well. Indeed, as shown in FIG. 2, the correlation (R2) is greater than 0.98.

Accordingly, the present disclosure provides a reliable, rapid, real-time, and portable method for determining microbial growth in a water-based fluid within a water fluid well that can be performed on-site to permit rapid remedial actions, as needed.

Unless otherwise indicated, all numbers expressing quantities of ingredients, properties such as molecular weight, reaction conditions, and so forth used in the present specification and associated claims are to be understood as being modified in all instances by the term “about.” Accordingly, unless indicated to the contrary, the numerical parameters set forth in the following specification and attached claims are approximations that may vary depending upon the desired properties sought to be obtained by the embodiments of the present invention. At the very least, and not as an attempt to limit the application of the doctrine of equivalents to the scope of the claim, each numerical parameter should at least be construed in light of the number of reported significant digits and by applying ordinary rounding techniques.

One or more illustrative embodiments incorporating the invention embodiments disclosed herein are presented herein. Not all features of a physical implementation are described or shown in this application for the sake of clarity. It is understood that in the development of a physical embodiment incorporating the embodiments of the present invention, numerous implementation-specific decisions must be made to achieve the developer's goals, such as compliance with system-related, business-related, government-related and other constraints, which vary by implementation and from time to time. While a developer's efforts might be time-consuming, such efforts would be, nevertheless, a routine undertaking for those of ordinary skill in the art and having benefit of this disclosure.

While compositions and methods are described herein in terms of “comprising” various components or steps, the compositions and methods can also “consist essentially of” or “consist of” the various components and steps.

Therefore, the present invention is well adapted to attain the ends and advantages mentioned as well as those that are inherent therein. The particular embodiments disclosed above are illustrative only, as the present invention may be modified and practiced in different but equivalent manners apparent to those skilled in the art having the benefit of the teachings herein. Furthermore, no limitations are intended to the details of construction or design herein shown, other than as described in the claims below. It is therefore evident that the particular illustrative embodiments disclosed above may be altered, combined, or modified and all such variations are considered within the scope and spirit of the present invention. The invention illustratively disclosed herein suitably may be practiced in the absence of any element that is not specifically disclosed herein and/or any optional element disclosed herein. While compositions and methods are described in terms of “comprising,” “containing,” or “including” various components or steps, the compositions and methods can also “consist essentially of” or “consist of” the various components and steps. All numbers and ranges disclosed above may vary by some amount. Whenever a numerical range with a lower limit and an upper limit is disclosed, any number and any included range falling within the range is specifically disclosed. In particular, every range of values (of the form, “from about a to about b,” or, equivalently, “from approximately a to b,” or, equivalently, “from approximately a-b”) disclosed herein is to be understood to set forth every number and range encompassed within the broader range of values. Also, the terms in the claims have their plain, ordinary meaning unless otherwise explicitly and clearly defined by the patentee. Moreover, the indefinite articles “a” or “an,” as used in the claims, are defined herein to mean one or more than one of the elements that it introduces.

What is claimed is:

1. A method for monitoring total microbial concentration in a water fluid well comprising water-based fluid, the method comprising:
collecting a water-based fluid sample from the water fluid well;
mixing the sample with a liquid reagent comprising luciferase enzyme, thereby forming a sample-liquid reagent composition;
measuring photon release of the sample-liquid reagent composition mixture in a luminometer, wherein the photon release is proportional to an amount of adenosine-5′-triphosphate (ATP) in the sample, and the amount of ATP in the sample is proportional to the total microbial concentration of the water-based fluid in the water fluid well;
determining the total microbial concentration of the water-based fluid in the water fluid well;
assigning an ATP-based action level based on the total microbial concentration of the water-based fluid in the water fluid well; and
performing an action based on the assigned ATP-based action level.

2. The method of claim 1, wherein the water fluid well is a water disposal well.

3. The method of claim 1, wherein the water fluid well is a water injection well.

4. The method of claim 1, wherein the water fluid well is a water supply well.

5. The method of claim 1, wherein the water-based fluid is selected from the group consisting of fresh water, saltwater, brine, seawater, produced water, wastewater, treated wastewater, and any combination thereof.

6. The method of claim 1, wherein the luminometer is portable and collecting the sample, mixing the sample, measuring the photon release, assigning the ATP-based action level, and performing the action is performed in real-time, on-site at the water fluid well.

7. The method of claim 1, wherein the total microbial concentration comprises free-living bacteria, free-living archaea, particle-associated bacteria, particle-associated archaea, bacterial biofilm, archaeal biofilm, and any combination thereof.

8. The method of claim 1, wherein the total microbial concentration comprises sulfate-reducing bacteria.

9. The method of claim 1, wherein the total microbial concentration comprises sulfate-reducing archaea.

10. The method of claim 1, wherein the photon release measurement has a repeatable relative standard deviation in the range of 7.4% to 12.4%.

11. The method of claim 1, further comprising filtering the sample prior to the mixing.

12. The method of claim 1, wherein the total microbial concentration of the water-based fluid in the water fluid well is less than 25 pg/ATP mL, and the assigned ATP-based action level is a good control ATP-based action level.

13. The method of claim 12, wherein the action comprises repeating the steps of collecting, mixing, measuring, determining, assigning, and performing based on a subsequent water-based fluid from the water fluid well to determine the total microbial concentration of the water-based fluid in the water fluid well.

14. The method of claim 1, wherein the total microbial concentration of the water-based fluid in the water fluid well is in the range of 25 pg/ATP mL to 200 pg/ATP mL, and the assigned ATP-based action level is a preventative action ATP-based action level.

15. The method of claim 14, wherein the action comprises increasing a frequency of monitoring the total microbial concentration of the water-based fluid in the water fluid well.

16. The method of claim 14, wherein the action comprises introducing a biocide into the water fluid well.

17. The method of claim 1, wherein the total microbial concentration of the water-based fluid in the water fluid well is greater than 200 pg/ATP mL, and the assigned ATP-based action level is a corrective action ATP-based action level.

18. The method of claim 17, wherein the action comprises introducing a biocide into the water fluid well.

19. The method of claim 18, wherein the biocide is selected from the group consisting of an oxidizing biocide, a non-oxidizing biocide, and any combination thereof.

20. The method of claim 17, wherein the action comprises identifying a source of microbial contamination, the source selected from the group consisting of a filter, a heat exchanger, a length of flow pipe, and the like, and any combination thereof.
